(19) United States
(12) Patent Application Publication
(10) Pub. No.: US 2003/0162848 A1
(43) Pub. Date: Aug. 28, 2003

(54) Reactor comprising a packed bed of supported catalyst or supported catalyst precursor, and a use of the reactor

(76) Inventors: Frederik Willem Hendrik Gimpel, CM Amsterdam (NL); Robert Martijn Van Hardeveld, CM Amsterdam (NL); Carolus Matthias Anna Maria Mesters, CM Amsterdam (NL); Gerardus Petrus Lambertus Niesen, CM Amsterdam (NL); Franciscus Johannes Maria Schrauwen, CM Amsterdam (NL)

Correspondence Address: Richard F Lemuth, Shell Oil Company, Intellectual Property, P O Box 2463, Houston, TX, 77252-2463, US

(21) Appl. No.: 10/333,888

(22) PCT Filed: Jul. 11, 2001
(86) PCT No.: PCT/EP01/08019

(51) Int. Cl. B01J 8/02; B01J 35/02; C07C 27/06

(52) U.S. Cl. 518/716; 502/325; 422/211

(57) ABSTRACT

A reactor comprising a packed bed of supported catalyst or supported catalyst precursor wherein the supported cata-lyst or the supported catalyst precursor comprise an external surface comprising a catalytically active metal or a precursor compound thereof, and the packed bed has a void content of more than 50% v and a specific surface area of more than 10 cm²/cm³, which is calculated as the total external surface area of the particles relative to the bed volume; the use of the said reactor in a chemical conversion process; a process for preparing hydrocarbons from syngas, which process comprises contacting a mixture of carbon monoxide and hydrogen in the said reactor, on the understanding that the catalytically active metal is a Group VIII metal which is at least in part present in metallic form; a packed bed of catalyst particles or catalyst precursor particles; and a catalyst particle or catalyst precursor particle.

The present invention relates to a reactor comprising a packed bed of supported catalyst or supported catalyst precursor. The invention also relates to a use of the reactor, in particular the use of the reactor in a process for the preparation of hydrocarbons from synthesis gas.

The catalytic preparation of hydrocarbons from synthesis gas, i.e. a mixture of carbon monoxide and hydrogen, is well known in the art and is commonly referred to as Fischer-Tropsch synthesis.

Catalysts suitable for use in a Fischer-Tropsch synthesis process typically contain a catalytically active metal of Group VIII of the Periodic Table of the Elements (Handbook of Chemistry and Physics, 68th edition, CRC Press, 1987-1988) supported on a refractory oxide, such as alumina, titania, zirconia, silica or mixtures of such oxides. In particular, iron, nickel, cobalt and ruthenium are well known catalytically active metals for such catalysts. Reference may be made to EP-A-398420, EP-A-178008, EP-A-167215, EP-A-168894, EP-A-363537, EP-A-498976 and EP-A-71770.

In the Fischer-Tropsch synthesis, as in many other chemical reactions, the supported catalyst, the reactants and a diluent, if present, in contact with one another usually form a three phase system of gas, liquid and solid. Such three phase systems may be operated, for example, in a packed-bed reactor or in a slurry-bubble reactor. A packe-bed reactor may comprise a packed bed of solid, relatively coarse catalyst particles through which there is a flow of gas and liquid. A slurry-bubble reactor may comprise a continuous phase of liquid with the solid, relatively fine catalyst particles suspended therein and gaseous reactants flowing as bubbles through the liquid. Traditionally, the catalytically active metal is dispersed evenly throughout the catalyst particles.

There is a continuous interest in finding catalysts and catalyst systems for use in the Fischer-Tropsch synthesis which provide an improved activity and an improved selectivity in the conversion of carbon monoxide into valuable hydrocarbons, in particular hydrocarbons containing 5 or more carbon atoms (“C₅+ hydrocarbons” hereinafter), and which minimise the formation of methane, which is a hydrocarbon carbon frequently considered as being of lower value.

US-A-5545674 discusses the use in the Fischer-Tropsch synthesis of catalysts which have a short diffusion length, i.e. they are low in diffusion limitation. Such catalysts may have the form of a fine powder for use in a slurry-bubble reactor, or they may be in the form of so-called shell catalysts. The shell catalysts comprise relatively coarse catalyst particles which contain the catalytically active metal positioned exclusively in a thin outer layer of the particles, instead of in an even distribution throughout the particles. The shell catalysts are primarily of interest for use in a packed-bed reactor. Compared with the traditional catalysts, the catalysts which have a short diffusion length exhibit a relatively high selectivity with respect to the formation of C₅+ hydrocarbons, and they suppress the production of methane.

As indicated hereinbefore, catalysts which have the form of a relatively fine powder can suitably be used in a slurry-bubble reactor. However, operational difficulties occur when the shell catalysts are used in a packed-bed reactor. Namely, given the fact that in the shell catalysts the catalytically active metal is present only in the outer layer of the catalyst particles, the quantity of catalytically active metal present in the reactor is relatively low, which causes that the reactor has a relatively low productivity, relatively to the reactor volume, when other process parameters are kept unchanged. This situation can not be improved satisfactorily by exploring the traditional catalyst shapes, such as beads or spheres, extrudates, saddles or the like. If one would choose to increase the quantity of catalytically active metal present, e.g. by decreasing the size of the catalyst particles, one would run into problems associated with a high pressure drop over the catalyst bed.

More generally, these problems occur in any chemical conversion process which involves a gas or liquid flow and in which diffusion limitation plays a role in relation to a solid catalyst. Thus, when put in a more general context, it is desirable to find a solution for the problem of using a shell catalyst in a packed-bed reactor, in an economically attractive operation.

As a solution to the stated problem, the present invention provides a reactor comprising a packed bed of supported catalyst or supported catalyst precursor wherein the supported catalyst or the supported catalyst precursor comprise an external surface comprising a catalytically active metal or a precursor compound thereof, and the packed bed has a void content of more than 50% v and a specific surface area of more than 10 cm²/cm³, which is calculated as the total external surface area of the particles relative to the bed volume.

When operated in a chemical conversion process which involves a gas or liquid flow, a packed bed as defined in accordance with this invention provides an improved, low pressure drop over the packed bed and an improved, high reactor productivity. In accordance with a preferred embodiment of this invention, this can be achieved by employing a packed bed of catalyst particles or catalyst precursor particles which are relatively thin and which have an extended shape, in particular particles which are bent to some extent, such as shavings and pieces of bent wire or bent tape. The invention is preferably carried out as a fixed bed multitubular reactor.

The invention further provides the use of a reactor in accordance with this invention in a chemical conversion process, in particular a process for preparing hydrocarbons from syngas, in which the catalytically active metal is a Group VIII metal which is present at least partly in metallic form.

The packed bed to be used in the present invention is suitably a supported catalyst in the form of a bed of solid, relatively coarse particles, or in the form of fixed structures (or arranged packings) as gauzes, corrugated sheet material which may or may not be perforated with holes, woven or non-woven structures, honeycombs and foams. For a general discussion about packed columns, especially arranged packings, reference is made to Perry's Chemical Engineer's Handbook (1984), 50th edition, 18-19 to 18-41. The reactor is especially suitable for downward gas/liquid flow reactions.

In addition, in preferred embodiments as specified in the claims hereinafter, the invention provides a packed bed of catalyst particles or catalyst precursor particles, a catalyst particle per se and a catalyst precursor particle per se, as well as to a packed bed comprising fixed structures or arranged packings comprising the catalyst or catalyst precursor, for example in the form of a coating and to fixed structures or arranged packings per se.

The skilled person will immediately appreciate that the invention obviates also disadvantages of a process operated in a slurry-bubble column. Namely, operation in a slurry-bubble column requires means to achieve and maintain a homogenous distribution of the catalyst over the entire liquid volume, and there is the need of separation of the reaction product from the relatively fine catalyst powder particles.

The void content of the catalyst bed is suitably at most 95% v, preferably at most 90% v. Suitably the void content is at least 55% v, preferably at least 60% v, in particular at least 65% v.

Suitably, the specific surface area of the packed bed is at least 15 cm²/cm³, more suitably at least 20 cm²/cm³, in particular at least 25 cm²/cm³, calculated as the total external surface area of the particles relative to the bed volume. Suitably, the specific surface area of the catalyst bed is at most 500 cm²/cm³, in particular at most 300 cm²/cm³, on the same basis. The specific surface area of the packed bed relates to the external, i.e. macroscopic surface area of the individual particles present in the packed bed, as opposed to their internal, i.e. microscopic surface area.

The supported catalyst may comprise particles and/or fixed structures or arranged packings. Usually the structures or packings will comprise an inert kernal (e.g. a commercially available gauze, corrugated place or (non)-woven structure) covered by a layer of catalyst or catalyst precursor. The particles may contain an inert kernal or be in the form of a homogeneous particle, i.e. the outer surface layer as well as the kernal comprising catalytically active material or precursor thereof.

The shape of the particles or the packings is not material to the invention, as long as—upon dumping or placing into a reactor—the particles or the packings form a packed bed in accordance with this invention. The skilled person will appreciate that in the packed bed so formed the voids are homogeneously distributed over the whole bed, i.e. without large empty spaces and without areas which do not have voids. For example, particles which are not free-flowing are not so easily dumped into a reactor and large empty spaces in the packed bed may result. Also particles which can easily stack are less preferred as they may cause the formation of areas which do not have voids, which leads to a higher pressure drop over the packed bed.

As indicated hereinbefore, in a preferred embodiment the packed bed comprises particles which are relatively thin and have an extended shape. In particular they are bent to some extent because this causes that the void content of the packed bed will be larger. Too much bending is less preferred and it is less preferred that the particles are branched. Namely, too much bending and branching would lead to a loss of free-flowability of the particles.

Suitably, the relatively thin and extended particles have a length, i.e. the largest dimension of the particles, of at least 1 mm, in particular at least 2 mm. Suitably, the relatively thin and extended particles have a length of at most 50 mm, in particular at most 25 mm. The relatively thin and extended particles may be bent and/or distorted, for example at two or more discrete locations, in one more directions. If the particles are bent and/or distorted, their length is deemed to be the length of the same particles after they have been straightened out. The relatively thin and extended particles may have a cross-section of any shape. Typical shapes are rectangular, oval and circular.

The aspect ratio of the relatively thin and extended particles is herein defined as the ratio of their length to their quotient of the volume over the external surface area. Typically the aspect ratio is at least 10 and typically at most 1000, more preferably in the range of from 20 to 500. Independently of this criterion, the relatively thin and extended particles, whether bent or not, fit within the boundaries of a hypothetical cylinder of which the length is the length of the particles as defined hereinbefore, and of which the ratio of the length and the diameter of the circular cross-section is typically at least 2, preferably at least 3, and typically at most 100. More preferably, the ratio is in the range of from 4 to 50.

The specifications of the particles as given in the previous two paragraphs apply when all particles have the same dimensions and form. Frequently, the relatively thin and extended particles do not have the same dimensions and form, in which case it is preferred that at least 80%, in particular at least 90%, more in particular all individual particles meet the specifications as given.

The catalytically active metal or the precursor compound thereof may be evenly distributed throughout the catalyst particles. If this is the case, the void content and the specific surface area of the packed bed as defined hereinbefore imply that the dimensions of the catalyst particles or the catalyst precursor particles are such that they exhibit the characteristics of a catalyst which has a short diffusion length.

However, preferably, the catalyst particles or the precursor catalyst particles are particles of a shell catalyst, which shell catalyst particles comprise a core and an outer layer covering the core and which outer layer comprises the metal or the precursor compound thereof. The skilled person will appreciate that the core is preferably inert, or inactive relatively to the (potential) catalytic activity of the outer layer.

Independent of whether the catalytically active metal or the precursor compound thereof is evenly distributed throughout the catalyst particles, or the catalyst particles or the precursor catalyst particles are particles of a shell catalyst, it is preferred that the catalytically active metal or the precursor compound thereof is supported on a support.

The support is typically a material having a large internal surface area. For example, the internal surface area is at least 20 m²/g, especially at least 25 m²/g, and more specially at least 35 m²/g. Suitably the internal surface area is at most 400 m²/g, especially at most 200 m²/g. Preferably the internal surface area is in the range of from 40 m²/g to 100 m²/g. The internal surface areas as quoted herein are deemed to be BET surface areas measured in accordance with ASTM D3663-92.

The support may be for example a carbon support, but preferably it is a refractory oxide. Examples of suitable refractory oxides include silica, alumina, titania, zirconia or mixed oxides comprising silica, alumina, titania or zirconia, such as silica-alumina or physical mixtures such as a mixture of titania and silica. Preferably, the refractory oxide comprises titania, zirconia or mixtures thereof, in particular the refractory oxide is a titania or a zirconia.

According to a preferred embodiment, the refractory oxide comprising titania, zirconia or mixtures thereof, may further comprise up to 50% w of another refractory oxide, typically silica or alumina, based on the total weight of the refractory oxide. More preferably, the additional refractory oxide, if present, comprises up to 20% w, even more preferably up to 10% w, on the same basis.

The refractory oxide most preferably consists of titania, in particular titania which has been prepared in the absence of sulphur-containing compounds. An example of such a preparation method involves flame hydrolysis of titanium tetrachloride.

In accordance with this invention the catalyst particles or catalyst precursor particles comprise a catalytically active metal or a precursor compound of the catalytically active metal. Typically the metal is a Group VIII metal, as in many chemical reactions, such as Fischer-Tropsch synthesis and hydrogenations, a Group VIII metal catalyst may be used.

For use in the Fischer-Tropsch synthesis it is preferred that the Group VIII metal is selected from iron, nickel, cobalt and ruthenium. More preferably, cobalt or ruthenium is selected as the Group VIII metal, because cobalt based catalysts and ruthenium based catalysts give a relatively high yield of C₅+ hydrocarbons. Most preferably, cobalt is selected as the Group VIII metal. A further metal may be present in order to improve the activity of the catalyst or the selectivity of the conversion of synthesis gas into hydrocarbons. Suitable further metals may be selected from manganese, vanadium, zirconium and rhenium. A preferred further metal is manganese or vanadium, in particular manganese.

If the catalytically active metal or the precursor compound is supported on a support, the amount of metal, in particular Group VIII metal, present on the support may vary widely. Typically, when the catalyst is used in the Fischer-Tropsch synthesis, the amount is in the range of from 1 to 50% w of the metal, based on the weight of the metal relative to the weight of the catalyst particles, if the metal is evenly distributed, or relative to the weight of the outer layer, if the catalyst particles are shell catalyst particles. In accordance with the definitions given hereinbefore, the outer layer is deemed to be the layer at the periphery of the particle which comprises 90% of the catalytically active metal or the precursor compound. Preferred ranges are from 3 to 40% w, in particular from 5 to 30% w, on the same basis.

Generally, the Group VIII metal and the further metal, if present in the catalyst, are located in the catalyst particles or the catalyst precursor particles at the same locations. The atomic ratio of the Group VIII metal to the further metal is typically at least 5:1 and it is typically at most 200:1.

If a shell catalyst is used, the core comprises preferably a material with a low internal surface area, because the lower the internal surface area, the less will be the chance that the core itself exhibits catalytic activity. In accordance herewith, if the catalytically active metal or the precursor compound thereof is supported on a support, the internal surface area of the support is preferably larger than the internal surface area of the core. In general, the core will have an internal surface area of less than 20 m²/g, especially less than 10 m²/g and in particular less than 2 m²/g.

The core comprises frequently an inorganic material, such as a refractory oxide, a ceramic material, a metal or a carbon. Suitable refractory oxides for use as the core are silica, alumina, zirconia, magensia and titania, and mixtures thereof Silica and alumina are a preferred refractory oxides for use as the core.

The use of a core which is based on a metal, i.e. the core is of a metallic nature, may be advantageous because it provides a shell catalyst which is strong and which has a relatively high heat conductivity. A relatively high heat conductivity is advantageous when the shell catalyst is used in a process where a substantial quantity of heat needs to be transferred from or to the reaction mixture, such as in a Fischer-Tropsch synthesis process. Suitable metals are aluminium, iron, copper, titanium and mixtures comprising one or more of these metals, like steel and brass. Aluminium and mixtures comprising aluminium are preferred, for example mixtures which comprise at least 80% w aluminium, in particular at least 90% w aluminium. The latter mixtures comprise typically at most 99.9% w aluminium, or even at most 99.99% w aluminium. Aluminium containing mixtures may comprise from 0.01 to 5% w of contaminants or additives selected from, for example, magnesium, silicon, copper, manganese, zinc, chromium, zirconium and titanium.

The core may be partly or wholly of a carbon or of an organic material, such as a polymer or another resinous material. Examples of suitable organic materials are polystyrenes, polyolefins, celluloses, hydrocarbon resins and epoxy resins. The carbon or the organic material may be removed in a later stage, for example during a calcination step as described hereinafter, in which case hollow catalyst particles are obtained or catalyst particles which have a core of low density (e.g. a core having a foam structure). As a matter of definition, the removal of the core is deemed to be a replacement of the core by a core which is an empty space, and the resulting (partially) hollow catalyst particles continue to be species of a shell catalyst.

The surface of the core may be pre-treated to achieve a better adhesion of the outer layer to the core, in particular after the calcination step as described hereinafter. The surface of the core may be modified, e.g. by removing impurities or by covering the surface with a coating. Thus, the core may be washed with water or diluted acid, such as aqueous phosphoric acid; or treated with a refractory oxide sol, such as a silica sol or an alumina sol, or a paint, such as a zirconium oxide paint. If the core comprises a refractory oxide, it may be pre-treated by calcination, for example by heating at elevated temperature, preferably at a temperature between 400 and 750° C., more preferably between 450 and 650° C. The duration of the calcination is typically from 5 minutes to several hours, preferably from 15 minutes to 4 hours. Suitably, the calcination is carried out in an oxygen-containing atmosphere, preferably air.

It is not excluded that the shell metal catalyst comprises further components, in addition to those mentioned herein.

The skilled person will be aware that suitable methods are known in the art for depositing the catalytically active metal or the precursor compound thereof on a support. For example, supported catalysts and catalyst precursors may be made by the methods known from WO-99/34917, EP-A-455307, EP-A-510771 and EP-A-510772. These references deal with supports of titania, alumina, silica and zirconia, respectively.

The catalytically active metal and the further metal, if applicable, may be introduced onto the support in the same manner and together. The catalytically active metal and the further metal may be introduced in the form of a precursor compound. Such precursor compounds include salts, such as nitrates, carbonates and acetates, chelates, such as acetylacetonates and alkyl acetonates, hydroxides and oxides, and the metal itself. Generally, the calcination step, as described hereinafter, will effect that the precursor compounds of the metal will be converted into the corresponding metal oxide.

The supported catalysts and catalyst precursors may be obtained in the form of a spray dried powder, or in the form of extrudates, which may be milled to obtain a powder. The powder so obtained may be mixed with a diluent to make a slurry.

In a preferred embodiment the slurry is made by admixing the catalytically active metal, optionally the further metal, and/or precursor compounds thereof, the support and/or a precursor of the support with the diluent.

It may be advantageous to have a precursor compound of the support present in the slurry because it increases after calcination step as described hereinafter the strength of the supported catalyst and/or the adhesion of the outer layer to the core, if applicable.

The precursor compound of the support may be a compound which yields a refractory oxide in the calcination step as described hereinafter. The precursor compound of the support may or may not be soluble in the diluent. The precursor compound of the support may be an organic salt or complex compound, in particular having up to 20 carbon atoms. Examples of such salts and complex compounds are salts, such as acetates, propionates, citrates; chelates, such as acetylacetonates, alkyl acetoacetates and chelates with lactic-acid; alcoholates, such as ethylates, aminoethylates and isopropylates; and alkyl compounds, such as ethyl and isooctyl compounds. Alternatively, the precursor of the support is an inorganic compound, such as a hydroxide, or an inorganic salt, such as a halide. Refractory oxide paints frequently comprise a precursor compound of a refractory oxide.

As an example, suitable precursor compounds of titanium dioxide are tetraethyl titanate, isostearoyl titanate and octyleneglycol titanate and triethanolamine titanate. A very suitable compound, in particular for use in combination with water as the diluent, is the ammonium salt of lactic acid chelated titanate.

The diluent for making the slurry may be an organic diluent, such as a lower alcohol, a lower ketone, a lower ester, or a lower ether, for example ethanol, acetone, methyl ethyl ketone, ethyl acetate, diethyl ether or tetrahydrofuran. In this patent document, when the term “lower” is used in conjunction with an organic compound the term specifies that the organic compound has at most six carbon atoms, in particular four carbon atoms. More suitable diluents are aqueous diluents, such as a mixture of an organic diluent and water, preferably comprising at least 50% w of water and less than 50% w of organic diluent, based on the total weight of the diluent. Most suitably, water is used as the single diluent.

The slurry may be used for spray coating onto the particles of the core, for making a shell catalyst. A suitable method and apparatus for spraying the slurry onto the particles of the core is known from Arntz et al., in “Preparation of Catalysts IV”, B Delmon et al. (Eds.), Elsevier, 1987, p. 137 ff. It is also possible to wet the particles of the core with the diluent and subsequently contacting the wetted particles with the powder, by sprinkling or dusting the powder onto the wetted particles or by tumbling the wetted particles in the powder.

Alternatively, the slurry may be extruded to form catalyst particles or catalyst precursor particles which do not have a core, i.e. which have the catalytically active metal or the precursor compound substantially evenly distributed over the particles. Such extruded particles with an even distribution may also be obtained directly by the methods of WO-99/34917, EP-A-455307, EP-A-510771 and EP-A-510772.

In an alternative embodiment, shell catalyst may be made by surface impregnation, for example using a spraying method or an immersion method, such as disclosed in US-A-5545674, EP-A-178008 and EP-A-174696. When surface impregnation is applied, the core and the support of the outer layer are necessarily of the same material.

It is preferred that the catalyst particles or the catalyst precursor particles are subjected to a calcination step. The calcination step increases the hardness and the strength of the coating and the adhesion of the coating to the core. The calcination step involves heating at elevated temperature, preferably at a temperature between 400 and 750° C., more preferably between 450 and 650° C. The duration of the calcination step is typically from 5 minutes to several hours, preferably from 15 minutes to 4 hours. Suitably, the calcination step is carried out in an oxygen-containing atmosphere, preferably air.

The thickness of the outer layer of the shell catalyst particles, typically after the calcination step, is in the range of from 0.001 to 0.15 mm, preferably in the range of from 0.002 to 0.1 mm, in particular in the range of from 0.005 to 0.08 mm. The thickness of the outer layer of the shell catalyst particles is herein defined differently for the various types of shell catalyst particles. The thickness of the outer layer of a coated shell catalyst particle is defined as the quotient of the volume of the coating which contains the catalytically active metal and the external surface area of the core particle. The thickness of the outer layer of a surface impregnated shell catalyst particle is defined as the thickness (d) of a layer at the periphery of the particle which comprises 90% of the catalytically active metal and which layer is selected such that at any point at the inner side of the layer the shortest distance to the periphery of the particle is the same and equals d.

The thickness of the outer layer as specified in the previous paragraph applies when all particles have the same thickness of the outer layer. Frequently, the thickness of the outer layer is not the same for all particles, in which case it is preferred that at least 80%, in particular at least 90%, more in particular all individual particles meet these specifications.

The catalytically active volume in the packed bed (i.e. the total volume of the particles which contains the catalytically active metal or the precursor compound thereof, typically after the calcination step) is suitable in the range of from 5 to 50% v, preferably in the range of from 10 to 40% v, relative to the volume of the packed bed. In this context, the catalytically active volume of a surface impregnated shell catalyst is deemed to be the volume of the layer at the periphery of the particle having the thickness d. The catalytically active volume of a coated shell catalyst particle is the volume of the coating. When the catalytically active metal or the precursor compound thereof is evenly distributed throughout the particles, the catalytically active volume is the total volume of the particles.

The fixed structures or arranged packings to be used in the present invention are well known in the literature and often commercially available. These structures or packings are usually made of metals or metal alloys or in the form of ceramic foams/ceramic honeycombs. These structures or packings may be covered with a layer comprising catalytically active material or a precursor thereof in the way as described above. Preferred materials for the structures or packings are the same as for the shell catalyst described above.

The reactor comprises basically a vessel, which comprises appendages for feed inlet, product outlet, and internals, which can hold the packed bed in place. The reactor suitably comprises inlets and outlets for auxiliary chemicals, and means for heating and/or cooling the reactor and its contents. The reactor is suitably designed such that it withstands internal pressure. The vessel may be filled with the catalyst particles or catalyst precursor particles by dumping the particles into the vessel. A plurality of vessels may be present in the reactor, so that the reactor can hold a plurality of packed beds, for example 125000, or even up to 40000 or more. The reactor may be a multi-tubular reactor.

If desired, the calcination step may be carried out inside the reactor.

The dimensions of the packed bed may be as follows. The height of the packed bed is typically in the range of from 1 to 20 m. The dimensions perpendicular to the height are typically in the range of from 1 cm to 10 m. The ratio of the latter dimensions to the length of the catalyst particles is typically in the range of from 5 to 1000, preferably in the range of 7 to 500.

The reactor and the metal catalyst may be used in a process for the preparation of hydrocarbons from carbon monoxide and hydrogen. Typically, when in use in that process, the metal which is present on the catalyst is a Group VIII metal and, typically, at least part of the Group VIII metal is present in its metallic state.

Therefore, it is normally advantageous to activate the Group VIII metal catalyst prior to use by a reduction, in the presence of hydrogen at elevated temperature. If desired, the reduction may be carried out inside the reactor. Typically, the reduction involves treating the catalyst at a temperature in the range from 100 to 450° C., at elevated pressure, typically from 1 to 200 bar abs, frequently for 1 to 200 hours. Pure hydrogen may be used in the reduction, but it is usually preferred to apply a mixture of hydrogen and an inert gas, like nitrogen. The relative amount of hydrogen present in the mixture may range between 0.1 and 100% v.

According to a preferred embodiment of the reduction, the catalyst is brought to the desired temperature and pressure level in a nitrogen gas atmosphere. Subsequently, the catalyst is contacted with a gas mixture containing only a small amount of hydrogen gas, the rest being nitrogen gas. During the reduction, the relative amount of hydrogen gas in the gas mixture is gradually increased up to 50% v or even 100% v.

It may be preferred to activate the Group VIII metal catalyst in-situ, that is inside the reactor for the preparation of hydrocarbons from synthesis gas. WO-97/17137 describes an in-situ catalyst activation process which comprises contacting the catalyst in the presence of hydrocarbon liquid with a hydrogen-containing gas at a hydrogen partial pressure of at least 15 bar abs., preferably at least 20 bar abs., more preferably at least 30 bar abs. Typically, in this process the hydrogen partial pressure is at most 200 bar abs.

The process for the preparation of hydrocarbons from synthesis gas is typically carried out at a temperature in the range of from 125 to 350° C., preferably from 175 to 275° C. The pressure is typically in the range of from 5 to 150 bar abs., preferably from 5 to 80 bar abs., in particular from 5 to 50 bar abs.

Hydrogen and carbon monoxide (synthesis gas) is typically fed to the process at a molar ratio in the range from 0.7 to 2.5. Low hydrogen to carbon monoxide molar ratios will increase the C₅+ selectivity of the catalysts, i.e. the selectivity of the formation of C₅+ hydrocarbons.

The gas hourly space velocity (“GHSV” hereinafter) may vary within wide ranges and is typically in the range from 400 to 20000 Nl/l/h, more typically from 500 to 10000 Nl/l/h. The term “GHSV” is well known in the art, and relates to the gas per hour space velocity, i.e. the volume of synthesis gas in Nl (i.e. at the standard temperature of 0° C. and the standard pressure of 1 bar (100,000 Pa)) which is contacted in one hour with one litre of catalyst particles, i.e. excluding inter-particular void spaces. Preferably the gas hourly space velocity is chosen in the range from 500 to 5000 Nl/l/h.

The invention will now be illustrated further by means of the following Examples.

### EXAMPLE I

A precursor of a shell metal catalyst was prepared as follows.

A slurry was prepared by mixing and milling together commercially available titania powder (P25 ex. Degussa, BET surface area 50 m²/g (ASTM D3663-92)), commercially available co-precipitated cobalt/manganese hydroxide, commercially available lactic acid titanate ammonium salt (ex Dupont, available under the trademark TYZOR LA), a commercially available ceramic zirconium oxide paint (obtained from ZYP Coatings, type ZO) and water. The slurry contained 16% w cobalt and 1.0% w manganese, calculated as the weight of elemental cobalt and manganese, relative to the weight of the calcination residue which can be formed by drying and calcining the slurry in air at 800° C. for 2 hours.

Aluminium shavings (typical dimensions: 6 mm by 1 mm by 0.1 mm, aspect ratio about 120, bent to a curvature with 2 cm radius and distorted over up to 90 degrees) were washed with 25% w aqueous phosphoric acid and with water and dried. The slurry was spray-coated onto the treated aluminium shavings. The spray-coated shavings were dried at 120° C. for 2 hours and subsequently calcined in air at 500° C. for 2 hours. The average thickness of the coating after the calcination was 30 μm.

### EXAMPLE II

A precursor of a shell metal catalyst was prepared as follows.

Straight pieces of aluminium wire (length 4 mm, 0.26 mm diameter) were washed with 25% w aqueous phosphoric acid and with water and dried. The slurry of Example I was spray-coated onto the treated aluminium shavings. The spray-coated pieces were dried at 120 ° C. for 2 hours and subsequently calcined in air at 500 ° C. for 2 hours. The average thickness of the coating after the calcination was 30 μm.

### EXAMPLE III

Straight pieces of aluminium wire (length 4 mm, 0.5 mm diameter, dented at 1 mm intervals with 0.2 mm depth) were washed with 25% w aqueous phosphoric acid and with water and dried.

### EXAMPLE IV

The uncoated aluminium shaving and pieces of wire of Example I, II and III, and the shell metal catalyst precursors of Examples I and II were dumped in a tubular reactor having a diameter of 2.54 cm (1 inch). The void content (in % v), the specific surface area (cm²/cm³, external surface area of the particles relative to the bed volume), the catalytically active volume (% v) of the packed beds so prepared are given in Table I. The pressure drop (bar/m bed height), measured at a nitrogen gas flow of 32.5 Nl/h in model experiments using the uncoated particles, is also given in Table I.

TABLE I

| | | Specific | | |
|---|---|---|---|---|
| | Void | surface | Catalytically | Pressure |
| | content | area | active volume | drop |
| | (% v) | (cm²/cm³) | (% v) | (bar/m) |
| Example I, | 78 | 44 | | 0.13 |
| uncoated | | | | |
| Example I, | 69 | 39 | 11 | |
| coated | | | | |
| Example II, | 67 | 51 | | 0.18 |
| uncoated | | | | |
| Example II, | 63 | 46 | 12 | |
| coated | | | | |
| Example III | 54 | 37 | | |

### EXAMPLE V

A precursor of a shell metal catalyst was prepared as follows.

Aluminium shavings (typical dimensions 4 mm by 1 mm by 0.1 mm) were washed with 25% w aqueous phosphoric acid, and coated with a commercially available zirconium 10 oxide paint (obtained from ZYP Coatings, type ZO).

Subsequently, an aqueous slurry comprising finely dispersed commercially available cobalt hydroxide and a commercially available ammonium zirconium carbonate (MEL Chemicals, available under the trademark BACOTE 20) was spray coated onto the aluminium shavings. The slurry comprised 67% w cobalt, calculated as the weight of cobalt metal, relative to the weight of a calcination residue which can be formed by drying and calcining the slurry in air at 800° C. for 2 hours. The spray-coated shavings were dried at 120° C. for 2 hours and subsequently calcined in air at 500° C. for 2 hours. The average thickness of the coating after the calcination was 20 μm.

### EXAMPLE VI

The precursor shell metal catalysts prepared in Example V was converted into an active Fisher-Tropsch catalyst by reduction, and subsequently applied in a Fisher-Tropsch synthesis as follows.

A micro-flow reactor containing the catalyst precursor particles in the form of a fixed bed was heated to a temperature of 280° C., and pressurised with a continuous flow of nitrogen gas to a pressure of 1 bar abs. The catalyst precursor was reduced in-situ for 24 hours with a mixture of nitrogen and hydrogen gas. During reduction the relative amount of hydrogen in the mixture was gradually increased from 0% v to 100% v. The water concentration in the off-gas was kept below 3000 ppmv.

Following reduction, the preparation of hydrocarbons was carried out with a mixture of hydrogen and carbon monoxide at a H₂/CO ratio of 1.1:1 and a pressure of 32 bar abs. The GHSV was 795 Nl/l/h. The reaction temperature, expressed as the weighted average bed temperature, was 213° C. After 40 hours of operation, the space time yield, expressed as grammes hydrocarbon product per litre catalyst particles (including the voids between the particles) per hour; the selectivity of methane, expressed in % w of the total hydrocarbon product; the selectivity to hydrocarbons containing 5 or more carbon atoms (C₅+ selectivity), expressed as % w of the total hydrocarbon product; and the selectivity of carbon dioxide, expressed in % w of the total hydrocarbon product; were as set out in Table II.

TABLE II

| | |
|---|---|
| Space time yield, g/l/h | 92 |
| Selectivity CH₄, % w | 6.2 |
| C₅⁺ selectivity, % w | 84 |
| Selectivity CO₂, % w | 2.0 |

### EXAMPLE VII

Melt-spinned aluminium pins (length 5 mm, diameter 0.5 mm; Transmet Corporation, Columbus, Ohio, USA) were first washed with toluene and acetone. The pins were then washed with aqueous acid and with demineralized water, and dried.

A mix was made from 1827.2 g TiO₂ (P25 ex Degussa) with 896.7 g Co/Mn co-precipitate (molar ratio Mn/Co=6% at/at) and water. The mix was milled for 33 minutes.

A slurry was prepared from the above mix with water and Tyzor LA. HNO₃ was added to the slurry to reduce the pH to about 7.

A precursor shell metal catalyst was prepared by spraycoating the above slurry on the aforementioned pins. The coated pins were dried at 120° C. and calcined at 500° C.

The precursor shell metal catalyst was converted into an active Fischer-Tropsch catalyst by reduction, as described in Example VI.

Following the reduction, the catalyst was subsequently applied in a Fischer-Tropsch synthesis as follows. The preparation of hydrocarbons was carried out with a mixture of hydrogen and carbon monoxide at a H₂/CO ratio of about 1.3 and a pressure of 32 bar abs. The GHSV was 1579 Nl/l/h. The reaction temperature, expressed as the weighted average bed temperature, was 227° C. After 109 hours of operation, the space time yield, C₅+ selectivity and the selectivity to methane and CO₂, as defined in Example VI, were as listed in the following table:

| | |
|---|---|
| Space time yield, g/l/h | 198 |
| Selectivity CH₄, % w | 5.1 |
| C₅⁺ selectivity, % w | 90 |
| Selectivity CO₂, % w | 1.9 |

1. A reactor comprising a packed bed of supported catalyst or supported catalyst precursor wherein the supported catalyst or the supported catalyst precursor comprise an external surface comprising a catalytically active metal or a precursor compound thereof, and the packed bed has a void content of more than 50% v and a specific surface area of more than 10 cm²/cm³, which is calculated as the total external surface area of the particles relative to the bed volume.

2. A reactor as claimed in claim 1, characterised in that the void content of the catalyst bed is at least 60% v, in particular at least 65% v.

3. A reactor as claimed in claim 1 or 2, characterised in that the specific surface area of the packed bed is at least 20 cm²/cm³, in particular at least 25 cm²/cm³, calculated as the total external surface area of the support relative to the bed volume.

4. A reactor as claimed in any of claims 1 to 3, characterised in that the packed bed comprises particles which have a length of at least 1 mm, in particular at least 2 mm, and an aspect ratio of at least 10, in particular in the range of from 20 to 500, wherein the aspect ratio is defined as the ratio of the length to the quotient of the volume over the external surface area.

5. A reactor as claimed in any of claims 1 to 3, characterised in that the packed bed comprises particles which have a length of at least 1 mm, in particular at least 2 mm, and which fit within the boundaries of a hypothetical cylinder which has a length equal to the length of the particles, and of which the ratio of the length to the diameter of the circular cross-section is at least 2, in particular at least 3, more in particular in the range of from 4 to 50.

6. A reactor as claimed in any of claims 1-5, characterised in that the total volume of the support which contains the catalytically active metal or the precursor compound thereof is in the range of from 5 to 50%, in particular in the range of from 10 to 40% of the bed volume.

7. A reactor as claimed in any of claims 1-6, characterised in that the external surface of the supported catalyst or supported catalyst precursor comprises a Group VIII metal, in particular cobalt, as the metal, supported on a (second) support which is a refractory oxide, in particular selected from silica, alumina, titania, zirconia or mixed oxides comprising silica, alumina, titania or zirconia, such as silica-alumina or physical mixtures such as a mixture of titania and silica, preferably the Group VIII metal being present at least in part in the metallic form, and the (second) support suitably having the form of particles, extrudates or foam.

8. A reactor as claimed in any of claims 1-7, characterised in that the supported catalyst or the supported precursor catalyst are particles of a shell catalyst, which shell catalyst particles comprise a relatively inert core and an outer layer covering the core and which outer layer comprises the metal or the precursor compound, preferably the core being based on a metal, in particular selected from aluminium, iron, copper, titanium and mixtures comprising one or more of these metals, or, the catalyst being in the form of a fixed structure or arranged packing, preferably made from metal, the metal suitably having the form of gauzes, woven or non-woven structures, honeycombs or foams.

9. The use of a reactor as claimed in any of claims 1-8 in a chemical conversion process, preferably a process for preparing hydrocarbons from syngas, which process comprises contacting a mixture of carbon monoxide and hydrogen in a reactor as claimed in claim 7 or 8.

10. A packed bed of supported catalyst or supported catalyst precursor as defined in claim 7 or 8, or a catalyst particle or catalyst precursor particle which forms upon dumping in a reactor together with a plurality of identical particles a packed bed as defined in claim 7 or 8.
